(19) United States
(12) Patent Application Publication
(10) Pub. No.: US 2013/0144176 A1
(43) Pub. Date: Jun. 6, 2013

(54) NON-INVASIVE BLOOD PRESSURE SENSOR

(71) Applicant: Ryszard M. Lec, Philadelphia, PA (US)

(72) Inventor: Ryszard M. Lec, Philadelphia, PA (US)

(73) Assignee: Drexel University, Philadephia, PA (US)

(21) Appl. No.: 13/704,283

(22) PCT Filed: Jun. 24, 2011
(86) PCT No.: PCT/US11/41800

§ 371 Date: Feb. 26, 2013

Related U.S. Application Data
(60) Provisional application No. 61/397,698, filed on Jun. 25, 2010.

(51) Int. Cl. A61B 5/021

(52) U.S. Cl. CPC A61B 5/021
USPC 600/485

(57) ABSTRACT

A non-invasive pressure measurement device measures pressure of fluid in a conduit via a piezoelectric transducer positioned proximate to the conduit having a fluid passing therethrough. The piezoelectric transducer is in communication with the conduit via a plate member having a protrusion extending outward from one side of the member. In one application, the device continuously measures blood pressure without the need for an inflatable cuff. Also, the device can detect and measure heart beat pulses from the fluid and utilize heart beat pulse information to provide further characteristics pertaining to the fluid in the conduit.

### CROSS REFERENCE TO RELATED APPLICATIONS

The present application claims the benefit of priority to U.S. Application Ser. No. 61/397,698, filed Jun. 25, 2010, the entirety of which is incorporated herein by reference.

### BACKGROUND

Blood pressure measurement is a staple of the medical profession. Blood pressure measurement methods typically require the use of an inflatable cuff, and, as in the case of oscillometric methods, requires strict calibration of equipment used.

The auscultatory method uses a stethoscope and a sphygmomanometer. This method comprises an inflatable cuff placed around the upper arm at roughly the same height as the heart, attached to a mercury or aneroid manometer. The inflatable cuff is fitted, then inflated until the artery is completely occluded. Listening with the stethoscope to the brachial artery at the elbow, the listener slowly releases the pressure in the inflatable cuff. When blood just starts to flow in the artery, the turbulent flow creates a “whooshing” or pounding (first Korotkoff sound). The pressure at which this sound is first heard is the systolic blood pressure. The inflatable cuff pressure is further released until no sound can be heard (fifth Korotkoff sound), at the diastolic arterial pressure.

The auscultatory method, however, typically requires the presence of a medical professional and does not allow for continuous monitoring of the blood pressure of a patient. Further, the inflatable arm cuff and other elements of the system can be bulky and obtrusive.

Another method of measuring blood pressure is the oscillometric method. The equipment used in the oscillometric method is functionally similar to that of the auscultatory method, but the method uses an electronic pressure sensor instead of the stethoscope and an expert's ear. In practice, the pressure sensor is a calibrated electronic device with a numerical readout of blood pressure. In most cases a cuff is inflated and released by an electrically operated pump and valve, which can be fitted on the wrist (elevated to heart height), although the upper arm is preferred. Values of systolic and diastolic pressure are computed, not actually measured, and the computed results are displayed.

The cuff is inflated to a pressure initially in excess of the systolic arterial pressure, and then reduces to below diastolic pressure over a period of about 30 seconds. When blood flow is nil (inflatable cuff pressure exceeding systolic pressure) or unimpeded (inflatable cuff pressure below diastolic pressure), cuff pressure will be essentially constant. It is essential that the inflatable cuff size is correct: undersized cuffs can yield too high a pressure, whereas oversized cuffs yield too low a pressure. When blood flow is present, but restricted, the cuff pressure, which is monitored by the pressure sensor, will vary periodically in synchrony with the cyclic expansion and contraction of the brachial artery, i.e., it will oscillate.

The oscillometric method, like the auscultatory method requires the use of an inflatable cuff and has the further problem of calibration. Further, the oscillometric method typically takes individual blood pressure measurements and continuous monitoring of blood pressure generally not achievable with this method. The devices that use the oscillometric method also tend to be bulky and inaccurate.

Invasive methods of blood pressure measurements are also known in the art, however, these require either implantation or otherwise piercing the skin. They are typically performed by medical professionals and have discomfort and other potential complications associated with them.

### SUMMARY

A non-invasive blood pressure measurement system, in an example embodiment, comprises a piezoelectric transducer portion operatively supported by a plate member, wherein the plate member has a fixed protrusion extending from one side there from. When the protrusion is placed proximate to a conduit having a fluid passing there through, the transducer is in operative communication with the conduit. The piezoelectric transducer directly measures various characteristics of the fluid, such as pressure, blood pressure waveform, pulses, changes, and the like, and provides electronic signals indicative thereof. The electronic signals are correlated with the pressure in the conduit. In one embodiment, the conduit is an artery or a vein. In such an example, blood pressure corresponds to systolic and diastolic pressures, and a heart blood pressure waveform can provide information about the blood pressure of a user in a non-inflatable and non-invasive system.

In an embodiment, a blood pressure sensor is configured as a wristwatch. The wristwatch detects the blood pressure of a wearer and provides blood pressure information to the wearer. The wristwatch comprises a plate member having a protrusion extending from one side therefrom. A transducer is operatively supported by the plate member and is operatively coupled to the protrusion. When the wristwatch is affixed on the user, the protrusion is configured to ergonomically fit proximate to a vein or artery. When the protrusion is placed proximate to an artery or vein, the plate member communicates an indication of pressure in the artery or vein to the transducer. In addition, the protrusion provides an intended pre-stress on the artery thus creating the intended mechanical boundary conditions and interacting in programmable manner with the blood pressure and blood flow. The transducer converts the indication of pressure into corresponding electrical signals, which are correlated with the pressure in the artery or vein. The wristwatch can comprise one or more systems configured to correlate the output of the piezoelectric transducer with the pressure in the conduit. The wristwatch also can comprise a display, alignment systems, wireless or wired connections, computing environments, timepieces, thermometers, gyroscopes, accelerometers and the like.

In another embodiment, a pressure system can be configured as an attachment to a different body part, or as an attachment to an animal, or to a mechanical conduit in biological, consumer or industrial systems.

The piezoelectric transducer in the blood pressure system can operate in one or more modes, such as, for example a passive mode and/or active modes. In passive mode, the output of the transducer can be monitored for an electronic signal generated directly by the transducer itself that can be correlated with the pressure in the conduit. In active mode operation, the output of the transducer can be measured for variations from operation in the selected active mode to determine changes in pressure based on changes in the fluid stream.

According to another embodiment, the blood pressure system comprises an array of transducers. The array of piezoelectric transducers provides alignment information and/or measure the pressure in the conduit. In one embodiment, for example, the individual outputs from each transducer of the array of transducers are used to determine the location and orientation of the conduit. In another embodiment, the outputs from the array are combined to determine pressure in the conduit. The piezoelectric transducers in the array can operate in passive or active modes, or their combinations. such as, for example, direct passive modes and active resonant modes.

In another example embodiment, the protrusion provides an ergonomic function, such that a wearer of the blood pressure system configured as a wrist watch will be able to feel when the protrusion is properly placed in proximity to a vein or artery. As an example, the protrusion can be configured such that it fits between the tendons on a user's wrist. As another example, the protrusion can be configured such that some discomfort may be felt by a user if the protrusion is not located properly.

In another embodiment, the protrusion is configured to maintain a location proximate to a vein or artery once it is in place proximate to the vein or artery. For example, if the protrusion is configured to fit between the tendons of the wrist of a user, the protrusion may be sized and shaped such that pressure from the tendons and friction of the skin would work to keep the protrusion in place proximate to the artery or vein.

In yet another example embodiment, the protrusion can deform the conduit in one or more ways such that the surface area of the conduit operatively coupled to the transducer can be increased. The protrusion can apply a programmable pre-stress on the artery influencing in desired manner with the blood pressure and blood flow. For example, a conduit such as artery may initially be cylindrical. The protrusion can be positioned proximate to the artery such that it presses on the artery, flattening it or otherwise increasing the surface area of the artery coupled to the protrusion thus creating the appropriate mechanical boundary for measuring blood pressure. Generally, the protrusion could be of various shapes, both regular and non-regular, and that shapes can undergo changes in real time. In addition, the protrusion could be fixed, removable and/or replaceable.

In an embodiment, the elastic member has a programmable elasticity and the geometry determined by a specific operation. The member elasticity does influence the mechanical conditions (boundary conditions) that rule the transfer of blood pressure from the artery to the transducer. The elasticity may vary from very stiff like metal to very soft like polymer depending on actual applications. The geometry could be almost of any shape that is determined by the specific applications as well. In some applications piezoelectric transducer and the elastic member can be integrated forming one functional element using, for example, micromachining fabrication technologies.

### BRIEF DESCRIPTION OF THE DRAWINGS

FIG. 1 depicts a blood pressure system configures as a wristwatch.

FIG. 2 depicts a cross sectional area of the blood pressure sensor.

FIG. 3 depicts an example of elements in a blood pressure system.

FIGS. 4a-4b depict example arrays of transducers.

FIGS. 5a-5b depicts the output of an array of transducers applied to a matrix.

FIG. 6 depicts an example flow diagram for detecting pressure with a blood pressure system.

FIG. 7 depicts an example flow diagram for measuring blood pressure with a blood pressure system.

FIG. 8 depicts an example block diagram of a blood pressure system in communication with a computing environment.

### DETAILED DESCRIPTION OF ILLUSTRATIVE EMBODIMENTS

Blood pressure measurements can be performed without piercing the skin (non-invasively) and without the use of an inflatable cuff. These measurements may be performed in a substantially continuous manner. Blood pressure measurements can be performed using a piezoelectric transducer wherein the piezoelectric transducer can be operatively coupled with an artery. The piezoelectric transducer can be able to measure heart beat pulses, and/or the waveform of the pressure in an artery or vein and correlate the pulses with the blood pressure. These waveforms and pulses can be continuously monitored thus providing, in addition to the magnitude of blood pressure, the information on important cardiovascular conditions such as arrhythmia, incoming heart attack or the response of a patient to the given medical treatment as well on physiological persons conditions like anxiety or panic attack.

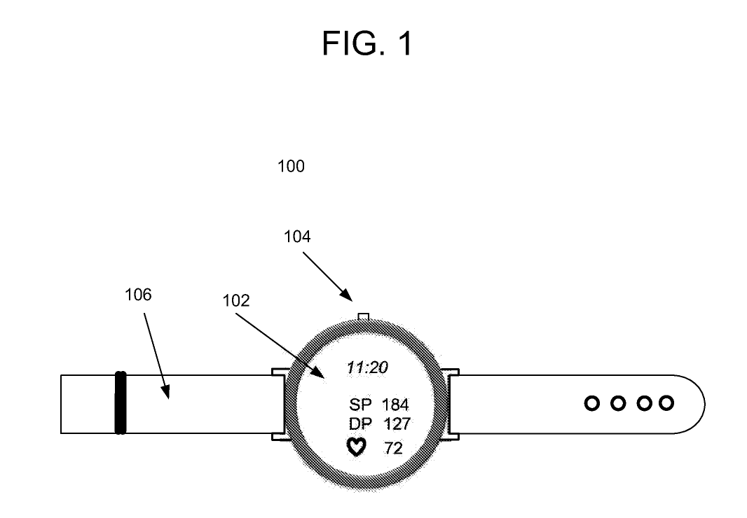

FIG. 1 depicts an example configuration for a blood pressure system 100 where the blood pressure system is configured as a wristwatch. The blood pressure system 100 can be used to measure and/or monitor information about a wearer, such as, for example, the blood pressure, heart rate, temperature and the like. Information about the wearer of the watch can be displayed to the wearer, or it may be provided to a third party. The blood pressure system 100 can also comprise other elements, such as, for example, a time piece, a wireless connection, power source, a calendar, or the like.

In one embodiment, the blood pressure system 100 can comprise a single platform, such as, for example a wristwatch. In such an embodiment, aspects of the user such as the blood pressure, temperature, heart rate, and the like can be determined from one or more contacts between the blood pressure system and the user. According to another embodiment, the blood pressure system 100 can be configured as a single unit attached to any body part, such as, for example, a wrist, a finger, arm, leg, chest, head foot, ankle toe or the like.

Elements of the blood pressure system 100 can be configured as one or more platforms attached to a user. For example, the blood pressure system 100 can comprise a watch and a ring. Blood pressure system 100 can comprise, without limitation, one or more of a ring, wrist mount, armband, ankle mount, leg mount, neck mount or the like.

Blood pressure system 100 can also be configured as two or more platforms, wherein a first platform is attached to a user, and a second platform is not attached to a user. For example, the blood pressure system 100 may be configured as a wristwatch attached via a wireless or wired connection to a phone or other portable electronic device, a home computer, a satellite receiver, a communications tower, a network, a server, or the like.

As shown in FIG. 1, the blood pressure system 100 can be configured as a wristwatch and can include a display 102. In one embodiment, blood pressure system 100 measures one or more of the blood pressure, blood pressure waveform, heart rate, temperature, and the like and provides this information to one or more elements of the blood pressure system, such as, for example, display 102. In one embodiment, the display 102 is a display that provides information, such as, for example, current blood pressure from blood pressure system 100. As another example, the display may provide a user with information from other elements of the blood pressure system or from outside sources, such as a wired or wireless connection, a clock and the like.

Blood pressure system can also perform the blood pressure waveform analysis that can utilized to advance cardiac diagnostic as well treatment capabilities. In one embodiment, the pressure waveform can be used to obtain information about the properties of a fluid flowing in a conduit such as an artery or vein. For example, waveform, measured over time may be correlated with the blood pressure in an artery or vein. Using the blood pressure wave form, and/or the blood pressure, elements of a patient health may be monitored. For example, events like incoming heart attack or the response of the watch user to the specific drug can be monitored. The continuous monitoring of the blood pressure, along with other aspects of an individuals such as temperature, heart rate and the like can allow for personalization of medical treatment.

Blood pressure and blood pressure waveform analysis taken over specific periods of recording times can be used to diagnose other non-cardiac medical conditions like liver, kidney, bladder, and other organs and physiological elements and systematic conditions.

The BP waveform analysis can be used to monitor treatment, optimize the dose of medications, and check a patient responsiveness to the specific drug and treatment procedures. Next, the BP sensor can be used to warn a user of coming health problems like arrhythmia, heart attack or kidney failure.

The display 102 may comprise any type of display known in the art, for example, without limitation, a digital display, a plasma screen, a touch screen, an LCD screen, a liquid crystal display, a clock face with hands or the like.

The display can be configured to provide the user with several pieces of information at a single time, such as, for example, time, pressure waveform, heart rate, temperature, current blood pressure, blood pressure trends, health status, alerts, medication requirements, time, date, and the like. According to another embodiment, the display may be configurable in any fashion. For example, an input 104 can be pressed or turned or otherwise activated to cause the display 102 to change appearance, show only one piece of information, show multiple pieces of information, and the like.

The blood pressure system 100 can contain any number of inputs 104 in any configuration that can be used as an input to alter one or more characteristics of the display or blood pressure system 100. For example, input 104 can be on the face of the sensor, on the back or side of the system. The input 104 may be configured as a switch, a knob, a keyboard or keypad, a touch pad, a touch screen or the like. In another embodiment, the blood pressure system 100 can comprise a receiver element that can allow for remote input. Input 104 may comprise a remote input element, such as, for example, input from a remote computing environment, input from a portable electronic device or the like.

According to one embodiment, the display 102 may provide a user with other information, such as, for example alignment information. As one example with the blood pressure system 100 configured as a wristwatch, the display 102 may provide alignment information in the form of an arrow, directions to tighten, move left, right, up or down on the arm or the like.

The blood pressure system 100 can have a startup or default mode of operation. The blood pressure system 100 may be configurable at startup or during operation in any fashion using inputs 104 as described above.

The blood pressure system 100 can comprise an element to attach the blood pressure system 100 proximate to a conduit. The attachment element can comprise a strap, harness, clamp, adhesive, elastic band, a leather watch band, metal links or any other type of strap, harness or the like can be used to affix the sensor. In one embodiment, the blood pressure system 100 is attached to a user by an attachment element such as, for example, strap 106 such that a transducer element on blood pressure system 100 is in proximity to a conduit flowing a fluid stream. In one embodiment, the conduit flowing a fluid stream is an artery or a vein.

The strap can be integrated with various auxiliary sensors such a temperature, stress, strain, humidity, motion monitoring, sensors and the like, that provide additional information and data that can enhance the measurement capabilities of the watch as well improve the measurement procedure, accuracy and reproducibility of the watch as well to provide the input to the user when the expected measurement conditions are present.

The blood pressure system 100 may be mounted on a human. In another embodiment, a non invasive sensor can be mounted on an animal, such as, for example, a horse or a dog.

The blood pressure system 100 may be mounted on any fluidic system (liquids, gases) in various industrial, environmental, etc. systems for the purpose of testing, process control, safety, fluidic system operational conditions in vehicle, etc.

The blood pressure system 100 can comprise a piezoelectric transducer as described below. In one embodiment, the blood pressure system may be configured such that, when the piezoelectric transducer is placed in proximity to a conduit flowing a fluid stream, the piezoelectric transducer may receive an indication of pressure in the conduit and provide a signal to one or more elements of blood pressure system 100 such that the pressure in the conduit may be determined.

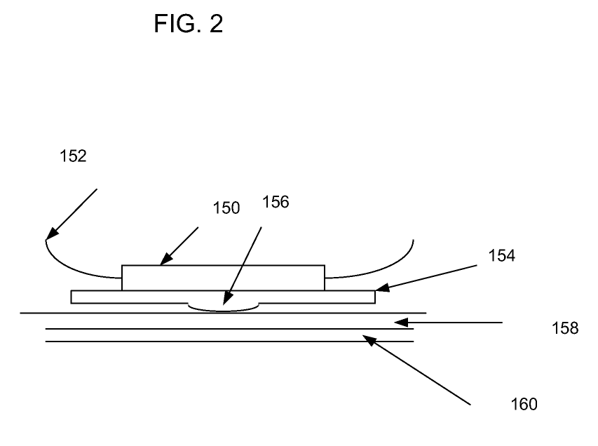

In the device shown in FIG. 1, the piezoelectric transducer can be located on the underside of the display 102. In another embodiment, the sensor can be located on the strap. In a further embodiment, the location of the piezoelectric transducer can be adjustable for each user, such that the display can be in a location and on the wrist preferred by the user. Further, if the non-invasive blood pressure sensor is affixed to a user on, for example, a finger, leg or the like, the piezoelectric transducer can be adapted such that the piezoelectric transducer is positioned proximate to an artery or vein. In one embodiment, the piezoelectric transducer is coupled to the conduit using a plate member having a protrusion extending from one side there from. Such an embodiment is described with respect to the blood pressure sensor in FIG. 2 described below.

In another embodiment the strap has a capability to measure a mechanical stress created by the tight/firmly fixed strap. This is achieved by integrating a stress sensor in the strap or use the blood pressure sensor itself or one of the sensors constituting the blood pressure sensor array.

Blood pressure system 100 can comprise a blood pressure sensor. FIG. 2 depicts an example embodiment of a blood pressure sensor with a piezoelectric transducer coupled to a conduit having a fluid stream passing there through. Piezoelectric transducer 150 can be a piezoelectric device, a capacitive device, a resistive device, a tactile transducer, a hydrophone or any other suitable pressure sensing device, including optical ones. The piezoelectric transducer can be comprised of any material suitable for the construction of a piezoelectric transducer and can be of any suitable shape and size, such as, for example, rectangular, cylindrical, amorphous, circular, elliptical, spherical or the like. The piezoelectric transducer can be of any thickness suitable for use in a non-invasive blood pressure device. In one embodiment, the piezoelectric transducer is sufficiently thin to fit in the wrist mounted device depicted above with respect to FIG. 2. Such piezoelectric transducer like bulk wave resonators, flexural modes, surface acoustic wave, surface skimming bulk wave, plate modes, and the like can be used for the purpose of blood pressure sensing.

The piezoelectric transducer 150 may be configured such that, when it is operatively supported by a plate member that is placed proximate to a conduit flowing a fluid stream 160, it receives an indication of pressure in the conduit 160 and provides a signal to the blood pressure system 100 for correlation with the pressure inside the conduit 160. In such an embodiment, the piezoelectric transducer 150 can be squeezed or otherwise affected by changes in pressure in the conduit flowing the fluid stream 160. The changes in pressure in the conduit 160 may be converted into an electrical output by piezoelectric transducer 150. One or more elements of blood pressure system 100 may correlate the electrical output of piezoelectric transducer 150 with the pressure in conduit 160. In one embodiment, the output of the piezoelectric transducer 150 is correlated with a pressure waveform indicative of the pressure in the conduit.

Piezoelectric transducer 150 can operate in one or more modes. In one embodiment, modes are defined by electrical signals provided to the piezoelectric transducer 150. For example, when an electrical signal is provided to piezoelectric transducer 150, piezoelectric transducer may vibrate or oscillate or otherwise respond mechanically to the electrical signal. Such an embodiment can be an active mode of operation of the transducer.

Piezoelectric transducer 150 can be in a passive mode. When piezoelectric transducer is in a passive mode, the piezoelectric transducer is not mechanically actuated by an electrical input from blood pressure system 100. Accordingly, when a piezoelectric transducer is in a passive mode and is coupled to a conduit, such as, for example, conduit 160, pressure changes in the conduit may cause one or more mechanical stresses or deformations on piezoelectric transducer 150 such that piezoelectric transducer 150 outputs an electrical signal. The output electrical signals may be provided to one or more elements of blood pressure system 100 and correlated with the pressure in the conduit 160. As an example, changes in pressure due to heart beats can be measured as an electrical output from, for example, a piezoelectric transducer 150. This output can be correlated with blood pressure, a pressure waveform, heart rate and the like.

In another embodiment, the piezoelectric transducer 150 can operate in one or more active modes. In such an embodiment, an electrical signal is provided to the piezoelectric transducer 150 by the blood pressure system 100, where the electrical signal can cause the piezoelectric transducer 150 to move, vibrate, oscillate, fluctuate, or the like. In an embodiment, the electronic signal provided to the piezoelectric transducer can set the oscillations of the piezoelectric transducer 150 at resonant modes of the piezoelectric transducer 150.

When the piezoelectric transducer 150 is in an active mode, the blood pressure system 100 may be provided an output signal indicative of the resonant mode from the piezoelectric transducer 150. If the piezoelectric transducer is coupled to a conduit flowing a fluid stream 160, information indicative of the pressure of the fluid stream may be communicated to the piezoelectric transducer 150. The change in pressure may cause the piezoelectric transducer 150 to vary in its resonant output such as frequency, amplitude, phase, velocity, attenuation, and the like. Changes in the resonant output may be observed by the blood pressure system 100 and may be correlated with the pressure in the conduit 160. As one example, the variations in the output can be correlated with one or more of the blood pressure, the heart rate or the like. In the above embodiments, electrical contacts 152 as described below can provide the piezoelectric transducer 150 with an electrical signal and can also have measurements of electrical signals coming from the transducer 150.

The piezoelectric transducers can be operated in single mode or multi-mode operation conditions such like thickness mode, lateral modes, and the like or multi-resonant excitation conditions. Multimode mode of operation can create a very versatile design approach to achieve sensing and measurement conditions control capabilities in simple sensing structures. In one embodiment, these features can be integrated in small size design using micromachining fabrication technologies. Such a structure, operating in both modes, can use the passive mode for measuring the dynamic blood pressure and the active mode for the static component of blood pressure, and their combination for providing more reliable and accurate blood pressure data.

The piezoelectric transducer 150 can have one or more electrical contacts 152 attached thereto. These electrical contacts 152 can provide a signal to the piezoelectric transducer 150. The electrical contacts 152 can receive a signal from the piezoelectric transducer 150, which can be filtered or otherwise conditioned by a circuit and used to correlate the output of the piezoelectric transducer 150 with the pressure in conduit 160. The electrical contacts 152 can be made of any material suitable for transmission of an electrical signal to and from a piezoelectric transducer 150. The electrical contacts 152 can be located on any surface of the piezoelectric transducer 150 such that the contacts can transmit electrical signals either to or from the transducer and can be of any thickness suitable for transmitting and receiving a signal from the piezoelectric transducer 150.

A plate member 154 can be situated in communication with the piezoelectric transducer 150 such that pulse information, pressure information, pressure waveform information, and the like from a heart beat is communicated from the plate member to the transducer. As shown in FIG. 2, in one embodiment, the piezoelectric transducer 150 can be mounted directly on the plate member 154. In another embodiment, one or more of the electrical contacts 152 can be located in between the piezoelectric transducer 150 and the plate member 154. As another example, one or more layers of any material can be located between the transducer piezoelectric 150 and the plate member 154. These layers can serve any purpose and can be of any thickness. Regardless of what lies between the plate member 154 and the piezoelectric transducer 150, these two elements should be in communication such that stresses in an artery from the heart beat of a subject are communicated to the piezoelectric transducer.

The plate member 154 member can be of any thickness to allow for the transmission of an indication of pressure between the conduit and the piezoelectric transducer 150. The thickness can also be any thickness that allows the plate member 154 to communicate a mode in a set of one or more modes of operation of the piezoelectric transducer 150 to the conduit. In one embodiment, the plate member is sufficiently thin to fit in a blood pressure sensor 100 configured as a wrist watch. The plate member may be flexible, or inflexible, or it may be plate, or any other shape fitting a given geometrical form of the watch.

The plate member 154 can be a rectangle, a cylinder, a cube, a sphere, an ellipsoid, have flat or curved surfaces or it can be amorphous in shape. The shape of the plate member 154 can have a protrusion extending from one side thereof and can provide an indication of pressure from a conduit to a piezoelectric transducer 150, and it can also provide an indication of a mode in a set of modes of operation for the piezoelectric transducer 150.

As an example, the stiffness or inflexibility of the plate member 154 can be related to the accuracy of the communication to the transducer. For example, in one embodiment, the plate member should be rigid such that a majority of the mechanical losses typically associated with, for example, a diaphragm or the like are prevented between the communication of the plate member 154 and the transducer 150. As another example, the plate member 154 should have a stiffness such that the piezoelectric transducer 150 can be operatively coupled to a conduit 160 having a fluid stream passing there through. In one embodiment, the plate member has a compression ratio In one embodiment, the Young modulus of the plate member can be from about 10 GPa to about 1000 GPa.

The blood pressure sensor can also comprise a protrusion. FIG. 2 depicts a protrusion 156. Protrusion 156 can extend from one side of the plate member 154. In one embodiment, the protrusion 156 is an ergonomic protrusion, such that a subject wearing a wrist mounted blood pressure sensor can know by the feeling of the protrusion 156 if the protrusion 156 is properly in place. In other words, the protrusion 156 may be configured to fit a body and can be used for alignment of the piezoelectric transducer 150. In another embodiment, the protrusion 156 can keep the piezoelectric transducer 150 in place. The dimensions of the protrusion can be such that the height of the protrusion extending from one side of the plate member can be from about 0.25 mm to about 10 mm, and the cross-section area of the protrusion can be from about 1 mm̂2 to about 100 mm̂2. For example, once the protrusion 156 is in place over an artery 160, the protrusion 156 can create pressure or otherwise be situated such that the transducer 150 remains substantially in communication with the artery 160. In a further embodiment, the fixed protrusion 156 can be used to deform the shape of an artery 160 such that a flattened and possibly large area of the artery can be in contact with the protrusion 156 and further to improve the communication between the piezoelectric transducer 150 and the artery 160.

In one embodiment, the protrusion can be dome shaped. In another embodiment, the protrusion can be a dome shape that is elongated in the direction parallel to the direction of the conduit. As one example, the protrusion can be sized and shaped such that it runs parallel with the artery of a subject's wrist and would fit between the tendons of a subjects wrist. As another example, two elongated protrusions, running parallel to an artery or vein may be situated such that the artery or vein is placed, squeezed, trapped or the like between the protrusions. In another embodiment, two protrusions of any shape may be situated such that an artery or vein can be situated, squeezed, placed, trapped or the like between them. here, two protrusions may be used to determine blood flow rate or blood flow velocity as well the mechanical features of an artery such as elasticity. In a further embodiment, each protrusion can be square, rectangular, triangular, many sided or amorphous in shape. In general, the protrusion or the system of protrusions is used to apply a programmable pre-stress or mechanical boundary conditions to the artery in order to optimize the blood pressure measurement process.

The protrusion or ergonomic member could be of any shape or shapes that can be fixed or undergo the change during the watch operation. In one embodiment, two dome shaped protrusion may be arranged such that each dome has a different height. As a further example, there may be two elongated protrusion or two dome protrusions of the same height, where each protrusion has a different base area or cross-sections. In a further embodiment, each protrusion can vary their size under the external pre-stress or push force, delivered automatically or by the finger of a user in order to apply a desired stress to the artery in order to calibrate or improve the measurements conditions, accuracy or inn order to address the variability of the wrist of the users.

The protrusion 156 can be situated proximate to a conduit 160 where one or more layers 158 are situated between the protrusion 156 and the conduit 160. As an example, the protrusion 156 can have one or more layers of skin between the protrusion 156 and the conduit. As another example, a disposable layer, a plastic sheath or the like can be situated between the protrusion 156 and the conduit. In such an example, if the blood pressure system 100 were to be used by several different users, a protective antibacterial layer may be placed between the user and the protrusion 156 of blood pressure system 100. The shape and size of the protrusion 156 can be substantially maintained regardless of any layers 158 that can come between it and the subject.

In another example embodiment, the disposable protrusion can be used to provide an auxiliary pre-testing and/or watch calibration functions for all measured parameters of interest like pressure, pulse rate, temperature, humidity, etc.

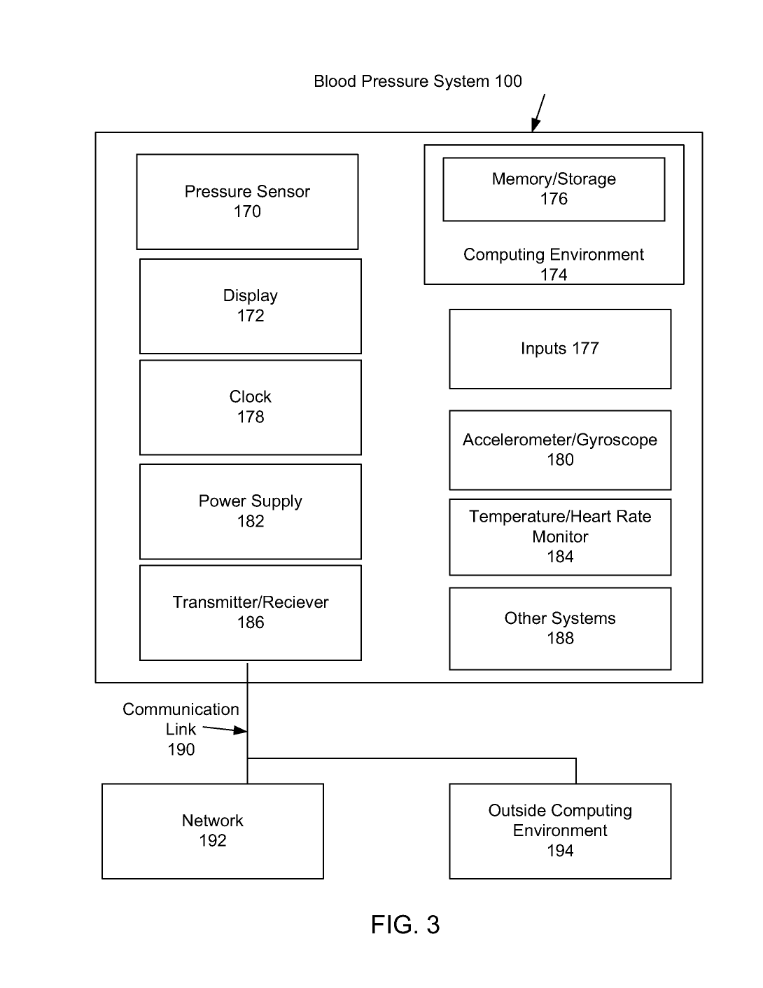

FIG. 3 depicts a block diagram of a blood pressure system 100, referenced with respect to FIG. 1. The block diagram depicts, one example of the plurality of elements that can be incorporated in a blood pressure system 100. Each individual element included in the drawing can or can not be included in specific implementations of blood pressure system 100.

Each element in blood pressure system 100 can be connected to the other elements in blood pressure system 100. For example, the elements of blood pressure system may be connected by wires, fibers, Bluetooth, RF, light pulses or otherwise such that signals, data, pulses and the like may be transmitted between the elements. FIG. 3 depicts several elements of blood pressure system 100 included in a single platform. In practice, the blood pressure system 100 can be a single platform, or it may have the elements of the system divided into a plurality of platforms. These platforms may be in connected via any manner known in the art including but not limited to wires, cables, wireless communication, low level magnetic, wi-fi, cellular, satellite, Bluetooth, RF transmissions and the like.

Blood pressure system 100 comprises a blood pressure sensor 170. The blood pressure sensor 170 can be the blood pressure sensor described above with respect to FIG. 2 or any other blood pressure sensor known in the art. The blood pressure sensor 170 can be used to test or monitor blood pressure. In one embodiment, the blood pressure sensor 170 can monitor blood pressure in a continuous or substantially continuous manner. In one embodiment, the blood pressure sensor of FIG. 2 may receive an indication of pressure in a conduit. The indication may be a force or motion exerted on the piezoelectric transducer by, for example, a heart beat. The piezoelectric transducer may convert the force into an electrical signal. The electrical signal may be provided to the other elements of the blood pressure system 100, such as the display 172, the computing environment 174, the memory or storage 176, other elements 188 or the transmitter 186. The electrical signal may be correlated with the pressure in the conduit 160 by one of the elements of the blood pressure system.

Blood pressure system 100 can comprise a display 172 which may be configured as display 102 discussed above with respect to FIG. 1. As one example, the display 172 can provide a subject with information such as blood pressure, temperature, time, heart rate, position, speed, device settings, warnings about a potential heart attack, arrhythmia, response to administered drug, or any other information available from blood pressure system 100. In addition, it can provide a standard advise or action steps like suggested administration of the drug or application of specific medical procedure. The display 172 can be any type of display known in the art, including but not limited to an LED, a plasma screen, a liquid crystal display, projector or any other display device known in the art. The display 172 can be configurable and can display information provided from the blood pressure system 100 and from communication link 190.

Blood pressure system 100 can comprise a computing environment 174, which can have memory 176 associated therewith. The computing environment 174 may be comprised of software and hardware elements in any configuration such that the computing environment 174 can be configured to control aspects of the blood pressure system 100. The computing environment 174 can comprise storage for data received on one or more of the elements of the blood pressure system 100 or from communications link 190. Memory 176 can also comprise one or more programs for use in monitoring aspects of a subject. The memory can also be used to store data associated with any other elements of blood pressure system 100.

Blood pressure system 100 can comprise one or more inputs 177. The inputs can allow for input on the blood pressure system. The inputs 177 can be any combination of a touch screen, keyboard, buttons, keypads, knobs, dials, switches and the like. The inputs may be used to configure the other elements of the blood pressure system 100, such as, for example, the color, brightness or content of the display, one or more alarms, the clock, content and processes on the computer environment, the pressure sensor, the temperature and heart rate monitors or any other element on the blood pressure system 100.

Input to the blood pressure system 100 may also come from outside computing environment 194 or network 192 via communication link 190. These inputs may configure or control one or more elements of the blood pressure system 100. For example, a remote input can be used to configure the other elements of the blood pressure system 100, such as, for example, the color, brightness or content of the display, one or more alarms, the clock, content and processes on the computer environment, the pressure sensor, the temperature and heart rate monitors or any other element on the blood pressure system 100.

Blood pressure system 100 can comprise a clock 178 and/or an accelerometer and/or gyroscope 180. In one embodiment, the accelerometer and/or a gyroscope 180 can be used to provide information about the motion of the blood pressure sensor or a user's body. As one example, movement of a user's body can cause peaks or valleys in blood pressure, which can lead to false alarms in a blood pressure system 100. An accelerometer or a gyroscope 180 or the like can be used to reduce the likelihood of a false reading, waveform, and the like. For example, if a user is moving their arm, the accelerometer 180 may send a signal to the computing environment 174 which may be used by the computing environment 174 to correct, alter or discard the readings from blood pressure sensor 170 during the movement of the arm.

Blood pressure system 100 can comprise a temperature monitor and/or a heart rate monitor 184. The blood pressure system 100 can comprise any method known in the art for monitoring the heart rate and temperature of a subject. The information from the temperature monitor and heart rate monitor 184 can be provided to the computing environment 174, the display 172, the computer memory 176, or other elements 188 in a blood pressure system 100. The temperature, heart rate and blood pressure may be used in conjunction to make one or more determinations about a user. For example, if the blood pressure, temperature and heart rate reach certain levels, an alarm or warning system may alert the user or a third party that the user is under a health risk or is in danger.

Blood pressure system 100 can comprise a power supply 182, which can provide power to the systems on the blood pressure system 100. The power supply can be a battery, a wall plug in, solar, or any other power supply system known in the art. In one embodiment, the electrical energy generated by the blood pressure signal can be used to charge the battery or other electrical storage systems.

In any of the embodiments, the non-invasive blood pressure sensor can comprise transmitter and/or receiver 186. The transmitter and receiver can transmit or receive information wirelessly or through a physical connection. For example, blood pressure information from the blood pressure system 100 may be transmitted to the home computer of a user, spouse, child, friend or the like. As a further example, the blood pressure information from blood pressure system 100 may be transmitted to a medical professional such as the user's doctor. Information may also be received on the blood pressure system 100 from any source, including a network 192 such as the internet, a home computer, or from a medical professional. In one embodiment, the blood pressure system sends blood pressure information to a medical professional who may review the information and provide treatment options, either immediate or long term to the user of the blood pressure system 100.

The transmitter/receiver may comprise any means known in the art for the wireless or wired transmission of data known. For example, low level magnetic, wi-fi, a phone line, cable connection computer port connection, RF, cellular, Bluetooth, satellite, edge, 3G, or any other means of communication may be used. Accordingly, information obtained about a subject from the sensor can be transmitted to any computing environment 194, such as, for example a home computer, a medical network, a phone, a watch with a display or any other suitable network 192.

Blood pressure system 100 can comprise other elements 188. For example, a GPS, an alarm or warning system, a telephone, or the like can be included in blood pressure system 100. As one example, a blood pressure system 100 can monitor a subject's blood pressure and his location. In one embodiment, if there is a spike or drop in blood pressure, heart rate, temperature or the like, the alarm can sound and a medical professional could be dispatched to the location designated by the GPS incorporated in the blood pressure system 100.

The blood pressure sensor 100 may comprise one or more piezoelectric transducers and one or more plate members. In one embodiment, multiple piezoelectric transducers can be positioned as an array in blood pressure sensor 100. An array of piezoelectric transducers can be configured to be operatively coupled to a single inflexible member. In another embodiment, each piezoelectric transducer in an array of transducers can be coupled to a plate member.

An array of piezoelectric transducers coupled to a plate member can have a single protrusion extending from one side of the plate member. The protrusion can have the same functionality as the protrusion 156 discussed above with respect to FIG. 2. In an embodiment where each piezoelectric transducer is operatively coupled to a plate member, each plate member may have a protrusion. In another embodiment, only a central member or main plate member may have a protrusion, while the other members do not have protrusion. In a further embodiment comprising an array of plate members, each member configured to be aligned along an axis of a conduit flowing a fluid may have a protrusion extending there from.

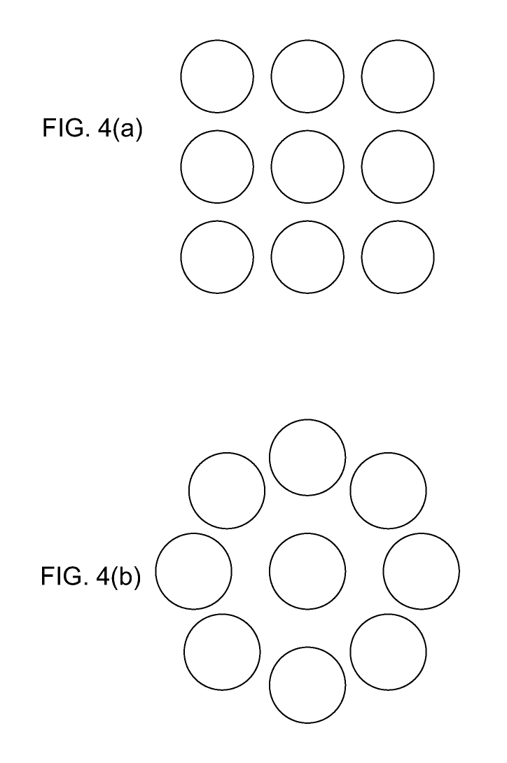

FIGS. 4(a)-4(b) depict arrays of transducers. As shown in FIGS. 4(a)-4(b), the transducers can be in a substantially rectangular array, or a substantially circular array. The transducers can be in any other array such as, for example, an amorphous array, an ellipse or the like. FIGS. 4(a)-4(b) depict circular transducers, however the transducers can be any other shape, including but not limited to rectangular, elliptical, amorphous or the like. The piezoelectric transducers can be any of the transducers 156 described above with respect to FIG. 2. In general both , the geometry of the transducer elements and the shape of deposited electrodes could vary from case-to-case addressing specific blood pressure sensing needs.

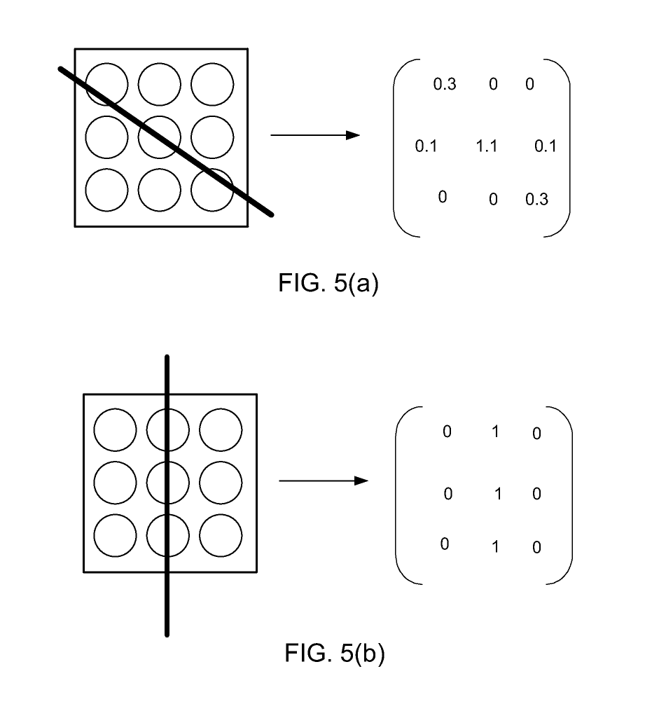

In one embodiment, the arrays of transducers can be used in the alignment of the non-invasive blood pressure sensor. For example, if there is a rectangular array of nine transducers and the three transducers parallel to the direction of flow all have the same pressure readings (although there may be a difference in the time that a pulse reaches each transducer), the system may be determine that the array is positioned parallel to the conduit. As a further example, if, in a rectangular array of 9 transducers with the central transducers positioned parallel to the conduit, and the three transducers to the left of the conduit and the three transducers the right of the conduit have the same output, then the blood pressure sensor may be positioned correctly in a direction perpendicular to the direction of flow of the conduit. FIG. 5 depicts a further example of this.

In another embodiment, the array can be used to determine the pressure in a conduit. In one embodiment, blood pressure may be determined using a single transducer in the manner described above with respect to FIG. 2. In another embodiment, blood pressure may be determined by correlating the collective outputs from the transducers in the array. For example, a mathematical algorithm such as the one described below with respect to FIG. 5 can be used to combine the outputs from each transducer in an array to determine pressure information. The information from the array of transducers can be provided to the elements of blood pressure system 100 described above with respect to FIG. 3 or to determine blood flow rate or blood flow velocity as well the mechanical features of an artery such as elasticity.

In another embodiment, the array can be used to determine other parameters and to take other measurements. In one embodiment, the array could be used to determine the acceleration by attaching a mass to one of the transducers such a transducer could be placed in a passive mode of operation. In such an embodiment, pressure created by forces acting on the mass attached to the transducer may cause the transducer to provide an electrical signal indicitivie of the motion of the blood pressure sensor, the wrist or the like to the blood pressure sensor. In another embodiment, one or more transducers can measure the temperature using a dynamic mode of operation. The same approach can be applied extending the use of the array for measurements of humidity, and other parameters of interest.

FIGS. 5a-5b depicts pressure information from an array of transducers depicted in a matrix. The matrixes in FIGS. 5(a)-(b) depict a normalized output from each transducer in a rectangular array of 9 transducers. The line across the array is a representation of a conduit. In FIG. 5(a), the conduit is not aligned parallel with the central axis of the central column of the array of transducers. The normalized output of the matrix can be used to determine that an artery is not properly aligned along the axis of the transducer, and what direction the array needs to move in order to properly align the array. Accordingly, alignment information may be provided such that the blood pressure sensor can be properly aligned. In FIG. 5(b), the artery has been aligned using the several transducers such that the artery crosses over the center of each transducer in the central column of the array of transducers.

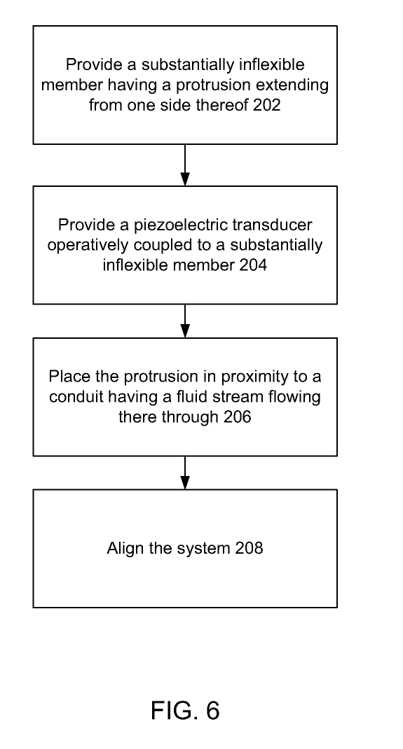

FIG. 6 depicts a process to detect blood pressure using a blood pressure system such as blood pressure system 100. At step 202 a blood pressure system may be provided with a plate member having a protrusion extending from one side thereof. The inflexible member and protrusion may be plate member 154 and protrusion 156 described above with respect to FIG. 2. As described above, the plate member and protrusion may be configured to provide an indication of pressure in a conduit to a transducer. Further, the plate member and protrusion may be configured such that coupling losses between the conduit and the transducer are kept to a minimum.

At step 204, a piezoelectric transducer may be operatively supported by the plate member in step 202. The piezoelectric transducer can be the piezoelectric transducer 150 discussed above with respect to FIG. 2. As discussed above, the piezoelectric transducer can be coupled to the plate member such that a force or indication of pressure can be received from the plate member by the piezoelectric transducer which can cause the transducer to output an electrical signal.

At step 206, the protrusion extending from one side of the plate member can be placed in proximity to a conduit having a fluid stream passing there through. The protrusion may have all of the functionality of protrusion 156 discussed above, including but not limited to ergonomic, alignment, location, affixing, deforming, detection, and the like. The protrusion may communicate an indication of pressure from the conduit to the piezoelectric transducer. Accordingly, the pressure in the conduit may be detected.

At step 208, the system may be aligned. The alignment may take place in any of the manners discussed above with respect to FIGS. 2, 4 and 5. For example, the protrusion may aid in alignment, or an array of transducers may aid in alignment. Alignment of the blood pressure system can allow for detection of pressure in a conduit.

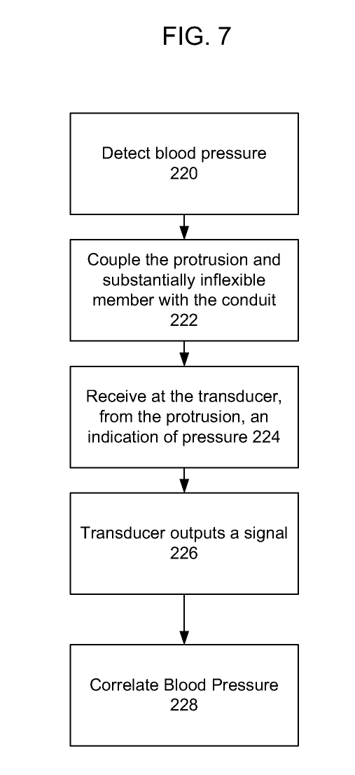

FIG. 7 depicts a process to measure blood pressure using a blood pressure system such at blood pressure system 100. At step 220 the blood pressure system may detect pressure in the conduit. The pressure can be detected using the flow diagram of FIG. 6. The detection of pressure can be used for further alignment of the blood pressure system, or it can be used to determine the blood pressure.

At step 222, the protrusion and the plate member are coupled with the conduit. When the plate member and the protrusion are coupled to the conduit, indications of pressure in the conduit may be transmitted to and through the protrusion and the plate member. As another example, when pressure in the conduit changes, the protrusion and the plate member may have a commensurate change in the indication that they receive and may provide to, for example, a piezoelectric transducer.

At step 224, an indication of pressure from the conduit, transmitted through the plate member may be received by the transducer. This indication may be in the form of a mechanical stress, an increase in force or the like. The indication can be commensurate with the pressure in the conduit.

A plate member, in some application, actually can exhibit am specific level of flexibility for the purpose of expanding the possible. In general, all presented discussions and comments could be applied to flexible member with appropriate adjustment of specific design in order to create the appropriate boundary conditions for the blood pressure measurements as well determination of other parameters.

At step 226, the piezoelectric transducer may output an electronic signal based on the indication of pressure received from the conduit. When the piezoelectric transducer is in an active mode described above with respect to FIG. 2, the output can be a variation from the input electronic signal. The output in a passive mode described above with respect to FIG. 2 can be a signal.

At step 228, the output signal may be transmitted to one or more of the elements of blood pressure system 100 described above with respect to FIG. 3. In one embodiment, the signal may be transmitted to a computing environment 174 or circuit that correlates the signal with the blood pressure, or the signal may be process and stored in a memory 176. The signal may be sent to the display 172, to a remote network or computing environment, or to any of the other elements on blood pressure sensor 100.

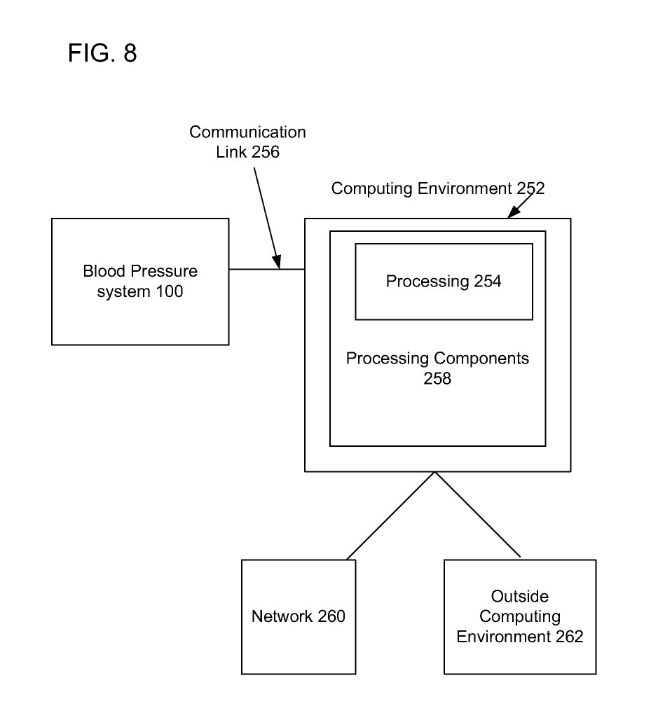

FIG. 8 depicts another embodiment of blood pressure system 100. The blood pressure system 100 can be implement as a divided platform where the blood pressure sensor comprises one or more of the elements described above with respect to FIG. 3 and is in communication with a computing environment 252. The communication link 256 between blood pressure sensor 250 and computing environment 252 can be a wireless link or a physical link, such as, for example, a cable, wire, low level magnetic, RF, wi-fi, Bluetooth, cellular satellite or the like. In one embodiment, the computing environment 252 can be a remote computing environment which can be used to provide information to the subject or a third party such as, for example, a spouse, medical professional, ambulance service or other center. The remote computing environment can be a personal computer, a phone, a server on a network, or any other computing environment known in the art. The computing environment 252 can be connected either wirelessly or via a physical connection with outside networks 260 such as the internet and/or other outside computing environments 262.

Data can be transferred from the blood pressure system 100 to the computing environment 252 as well as from the computing environment 252 to the blood pressure system 100. This data can comprise, without limitation, monitoring data, heart rate data, temperature data, settings for the blood pressure sensor, settings for one or more transducers, inputs to change the settings of the blood pressure sensor, tending data, warnings, corrections, updates in software, detection/diagnosis of various diseases, optimizing medical and/or drug treatment, personalizing the medical/drug treatments, or any other suitable data.

Computing environment 252 can comprise processing 254 that can be stored in processing components 258 on computing environment 252. The processing 254 can receive data from the blood pressure system 100 and can provide input to the blood pressure system 100. The processing 254 can process, transfer or store the data from blood pressure system 100. In one embodiment, the processing can be used to monitor the blood pressure of a user and provide warnings if the user's blood pressure rises above, or sinks below a certain level. In another embodiment, the blood pressure of the user can be monitored and stored such that a trend line or graph showing blood pressure over time can be created.

The processing 254 can also comprise temperature and heart rate monitoring elements. The combination of data comprising heart rate, temperature and blood pressure can be used in any way to provide information to a user or a third part. As one example, an athlete can be able to monitor his exertion and track it in software. As another example, the blood pressure software can comprise a function for use in a “love meter” or “mood” ring or watch. For example, a program can be created such that changes in blood pressure, heart rate and temperature are correlated with certain moods or physical responses and reported to a user for entertainment purposes or otherwise.

Additionally, the subject matter of the present disclosure includes combinations and subcombinations of the various processes, systems and configurations, and other features, functions, acts, and/or properties disclosed herein, as well as equivalents thereof.

1. A non-invasive pressure detection system comprising:
a plate member comprising a protrusion extending out from one side thereof; and
a piezoelectric transducer portion operatively supported by the plate member, and operatively coupled to the protrusion;
such that, when the protrusion is operatively coupled to a conduit having a fluid stream passing there through, the plate member is configured to receive an indication of the pressure of the fluid, and wherein the plate member is configured to provide the indication of the pressure of the fluid to the piezoelectric transducer portion, and wherein said piezoelectric transducer portion emits an electrical signal related to a pressure of the fluid in response to the indication of pressure from the plate member.

2. The system of claim 1, wherein the protrusion is a fixed ergonomic protrusion.

3. The system of claim 2, wherein the fixed ergonomic protrusion is configured to position the piezoelectric transducer portion on a wrist, according to the location of the conduit.

4. (canceled)

5. The system of claim 1, wherein the protrusion is sized and configured to remain substantially fixed with respect to the conduit.

6. The system of claim 1 wherein the protrusion is dome shaped.

7. (canceled)

8. The system of claim 7 wherein the protrusion has a length of about 1 mm to 20 mm and a width of about 1 mm to 20 mm and the height about a single mm to 16 mm.

9. (canceled)

10. The system of claim 1, further comprising at least one of an accelerometer, a gyroscope, a timepiece, a thermometer, a heart rate monitor, a transmitter, or a receiver.

11. The system of claim 1, further comprising one or more piezoelectric transducer portions attached to one or more plate members arranged as an array.

12. (canceled)

13. (canceled)

14. The system of claim 11 wherein said array is configured to position the piezoelectric transducer portion accordingly to the location of the conduit.

15. The system of claim 1 wherein a current supplied to the piezoelectric transducer portion is configured to operate the piezoelectric transducer portion in a mode in a set of one or more modes.

16. The system of claim 15 wherein the modes are resonant modes.

17. A method of non-invasively determining pressure in a conduit, the method comprising:
coupling a protrusion to a conduit having a fluid stream passing there through, wherein the protrusion extends out from one side of a plate member and wherein a piezoelectric transducer portion is operatively supported by the plate member;
receiving, at the transducer, an indication of pressure in the conduit;
emitting, from the transducer, an electric signal indicative of the pressure in the conduit; and
correlating an output of the piezoelectric transducer portion with the pressure in the conduit.

18. (canceled)

19. (canceled)

20. (canceled)

21. The method of claim 17, further comprising positioning the piezoelectric transducer portion with said protrusion

22. The method of claim 17 further comprising deforming the conduit with said protrusion.

23. (canceled)

24. (canceled)

25. (canceled)

26. (canceled)

27. The method of claim 17 wherein the plate member further comprises an array of piezoelectric transducer portions operatively coupled to the plate member.

28. (canceled)

29. (Canceled)

30. The method of claim 17, further comprising providing a current to the piezoelectric transducer portion to operate the piezoelectric transducer portion in a mode in a set of one or more modes.

31. The method of claim 30, wherein the modes are active modes.

32. The method of claim 30, wherein the modes are passive modes.

33. The method of claim 30 wherein the modes are combination of active and passive modes.

34. The method of claim 17 further comprising providing a portion of the electric signal from the transducer to charge a battery.
